(19) United States
(12) Patent Application Publication
(10) Pub. No.: US 2024/0234621 A1
(43) Pub. Date: Jul. 11, 2024

(54) DETECTION DEVICE AND MEASUREMENT DEVICE

(71) Applicant: SEIKO EPSON CORPORATION, Tokyo (JP)

(72) Inventors: Takashi TAJIRI, MATSUMOTO-SHI (JP); Takefumi FUKAGAWA, FUJIMI-MACHI (JP)

(73) Assignee: SEIKO EPSON CORPORATION, Tokyo (JP)

(21) Appl. No.: 18/403,684

(22) Filed: Jan. 3, 2024

(30) Foreign Application Priority Data
Jan. 6, 2023 (JP) 2023-000913

(51) Int. Cl. H01L 31/12; A61B 5/00; A61B 5/024; H01L 31/0232

(52) U.S. Cl. CPC H01L 31/125; A61B 5/02444; A61B 5/681; H01L 31/02327

(57) ABSTRACT

A detection device includes a first light emitting part that emits first light having a first wavelength band toward a living body, a second light emitting part that emits second light having a second wavelength band longer than the first wavelength band toward the living body, a first light receiving part that receives the first light emitted from the first light emitting part and radiated from the living body, a second light receiving part that receives the second light emitted from the second light emitting part and radiated from the living body, a first optical member that refracts the first light, and a second optical member that refracts the second light.

The present application is based on, and claims priority from JP Application Serial Number 2023-000913, filed Jan. 6, 2023, the disclosure of which is hereby incorporated by reference herein in its entirety.

### BACKGROUND

### 1. Technical Field

The present disclosure relates to a detection device and a measurement device.

### 2. Related Art

Various measuring techniques for non-invasively measuring biological information such as those using pulse waves have thus far been proposed. For example, JP-A-2020-171459 discloses a technique in which, in a detection device including a light emitting part that emits light to a living body and a light receiving part that receives the light emitted from the light emitting part and incident thereon by being reflected by the living body, a cover that covers the light emitting part and the light receiving part is configured of a Fresnel lens.

However, in the above-described detection device, one Fresnel lens is provided to cover a bottom surface of the detection device, and thus when light of a plurality of types of wavelength bands is emitted from the light emitting part, it is difficult to appropriately correct directivity of the light of each wavelength. For this reason, there is a problem in that the light from the light emitting part is reflected by a skin surface, and detection accuracy of the light receiving part is reduced.

### SUMMARY

According to one aspect of the present disclosure, provided is a detection device including a first light emitting part that emits a first light having a first wavelength band toward a living body, a second light emitting part that emits a second light having a second wavelength band longer than the first wavelength band toward the living body, a first light receiving part that receives the first light emitted from the first light emitting part and radiated from the living body, a second light receiving part that receives the second light emitted from the second light emitting part and radiated from the living body, a first optical member that refracts the first light, and a second optical member that refracts the second light.

### BRIEF DESCRIPTION OF THE DRAWINGS

FIG. 1 is a side view of a measurement device according to a first embodiment.

FIG. 2 is a configuration diagram focusing on functions of the measurement device.

FIG. 3 is a plan view of a detection device.

FIG. 4 is a cross-sectional view along line IV-IV in FIG. 3.

FIG. 5 is a diagram for describing operations of the detection device.

FIG. 6 is a plan view of a measurement device according to a second embodiment.

FIG. 7 is a cross-sectional view along line VII-VII in FIG. 6.

FIG. 8 is a plan view of a measurement device according to a third embodiment.

FIG. 9 is a plan view of a measurement device according to a fourth embodiment.

FIG. 10 is a plan view of a measurement device according to a fifth embodiment.

### DESCRIPTION OF EMBODIMENTS

Embodiments of the present disclosure will be described below with reference to the drawings. Also, in each of the following figures, the scales and angles of members are made different from those of actual ones in order to make each member have a recognizable size.

### First Embodiment

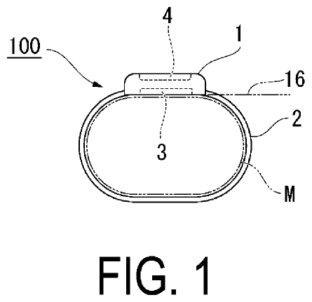

FIG. 1 is a side view of a measurement device 100 of a first embodiment. The measurement device 100 of the embodiment shown in FIG. 1 is a biometric device that non-invasively measures biological information of a subject (for example, a person), which is an example of a living body, and is attached to a site of the living body (hereinafter referred to as a “measurement site”) M serving as a measurement target in a body of the subject. The measurement device 100 of the embodiment is a wristwatch type portable device that includes a housing part 1 and a belt 2 and can be worn on a wrist of the subject, which is an example of the measurement site M, by wrapping the band-shaped belt 2 around the wrist. In the embodiment, pulse waves (for example, a pulse interval PPI) and an oxygen saturation (SpO2) of the subject are exemplified as the biological information. The pulse waves indicate changes over time in volume within blood vessels linked to the heart beat. The oxygen saturation indicates a proportion (%) of hemoglobin bound to oxygen in hemoglobin in blood of the subject and is an index for evaluating a respiratory function of the subject.

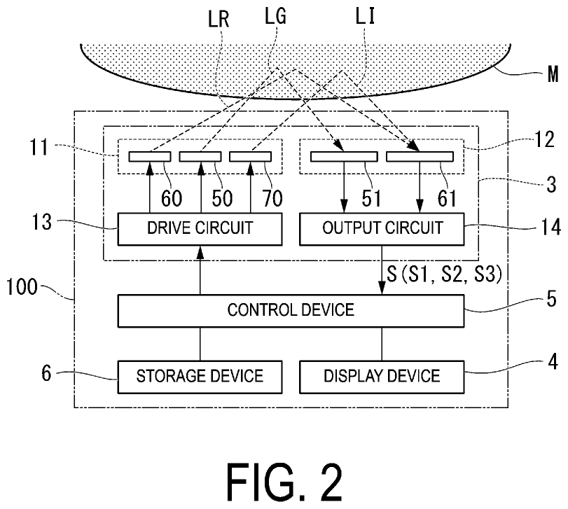

FIG. 2 is a configuration diagram focusing on functions of the measurement device 100. As shown in FIG. 2, the measurement device 100 of the embodiment includes a control device 5, a storage device 6, a display device 4, and a detection device 3. The control device 5 and the storage device 6 are installed inside the housing part 1. As shown in FIG. 1, the display device 4 is installed on a surface of the housing part 1 on a side opposite to the measurement site M and displays various images including measurement results under the control of the control device 5. The display device 4 is, for example, a liquid crystal display panel.

The detection device 3 is an optical sensor module that generates detection signals S in accordance with a state of the measurement site M. As shown in FIG. 1, the detection device 3 is installed, for example, on a surface (hereinafter referred to as a detection surface) 16 of the housing part 1 that faces the measurement site M. The detection surface 16 is a surface that comes into contact with the measurement site M. As shown in FIG. 2, the detection device 3 of the embodiment includes a light emitting unit part 11, a light receiving unit part 12, a drive circuit 13, and an output circuit 14. Also, one or both of the drive circuit 13 and the output circuit 14 can be installed as external circuits with respect to the detection device 3. That is, the drive circuit 13 and the output circuit 14 may be omitted from the detection device 3.

The light emitting unit part 11 includes a first light emitting part 50, a second light emitting part 60, and a third light emitting part 70. The first light emitting part 50, the second light emitting part 60, and the third light emitting part 70 are elements that emit light having different wavelengths to the measurement site M.

The first light emitting part 50 radiates, as first light having a first wavelength band, green light LG having a green wavelength band of 520 nm to 550 nm toward the measurement site M. The green light LG of the embodiment is, for example, light with a peak wavelength of 520 nm.

The second light emitting part 60 radiates, as second light having a second wavelength band longer than the first wavelength band, for example, red light LR having a red wavelength band of 600 nm to 800 nm toward the measurement site M. The red light LR of the embodiment is, for example, light with a peak wavelength of 660 nm.

The third light emitting part 70 radiates, as third light having a third wavelength band longer than the second wavelength band, for example, near-infrared light LI having a near-infrared wavelength band of 800 nm to 1300 nm toward the measurement site M. The near-infrared light LI of the embodiment is, for example, light with a peak wavelength of 905 nm.

For light emitting parts forming the first light emitting part 50, the second light emitting part 60, and the third light emitting part 70, for example, bare chip type or bullet type light emitting diodes (LEDs) are suitably used. Also, wavelengths of light radiated by the light emitting parts are not limited to the above numerical ranges. Hereinafter, unless the first light emitting part 50, the second light emitting part 60, and the third light emitting part 70 are particularly distinguished, they will be collectively referred to as the light emitting parts 50, 60, and 70.

The drive circuit 13 supplies a drive current to cause each of the light emitting parts 50, 60, and 70 to emit light. The drive circuit 13 of the embodiment periodically causes each of the light emitting parts 50, 60, and 70 to emit light in a time-division manner. The light radiated from the light emitting parts 50, 60, and 70 is incident on the measurement site M and propagates inside the measurement site M while being repeatedly reflected and scattered, and then is radiated to the housing part 1 side and reaches the light receiving unit part 12. That is, the detection device 3 of the embodiment is a reflection type optical sensor in which the light emitting unit part 11 and the light receiving unit part 12 are located on one side with respect to the measurement site M.

The light receiving unit part 12 receives the light arriving from the measurement site M due to the light emission from the light emitting unit part 11. The light receiving unit part 12 of the embodiment includes a first light receiving part 51 and a second light receiving part 61. The first light receiving part 51 and the second light receiving part 61 generate detection signals in accordance with an intensity of received light. Hereinafter, unless the first light receiving part 51 and the second light receiving part 61 are particularly distinguished, they will be collectively referred to as “the light receiving parts 51 and 61.”

The first light receiving part 51 receives the green light LG, which is radiated from the first light emitting part 50 and propagates in the measurement site M, and generates a detection signal in accordance with an intensity of received light. The second light receiving part 61 receives the red light LR, which is radiated from the second light emitting part 60 and propagates inside the measurement site M, or the near-infrared light LI, which is radiated from the third light emitting part 70 and propagates inside the measurement site M, and generates a detection signal in accordance with an intensity of received light.

The output circuit 14 is configured to include, for example, an A/D converter that converts detection signals generated by the light receiving parts 51 and 61 from analog to digital and an amplifier circuit that amplifies the converted detection signals (both not shown), and generates a plurality of detection signals S (S1, S2, and S3) corresponding to different wavelengths.

The detection signal S1 is a signal indicating the intensity of the light received by the first light receiving part 51 when the green light LG radiated from the light emitting part 50 is received. The detection signal S2 is a signal indicating the intensity of the light received by the second light receiving part 61 when the red light LR radiated from the second light emitting part 60 is received, and the detection signal S3 is a signal indicating the intensity of the light received by the second light receiving part 61 when the near-infrared light LI radiated from the third light emitting part 70 is received.

Generally, an amount of light absorbed by blood varies when blood vessels expand and contract, and thus each of the detection signals S becomes a pulse wave signal including a periodic fluctuation component corresponding to a pulsation component (a volume pulse wave) of an artery inside the measurement site M.

In addition, the drive circuit 13 and the output circuit 14 are mounted on a wiring board together with the light emitting unit part 11 and the light receiving unit part 12 in the form of an IC chip. Also, as described above, the drive circuit 13 and the output circuit 14 can be installed outside the detection device 3.

The control device 5 is an arithmetic processing device such as a central processing unit (CPU) or a field-programmable gate array (FPGA) and controls the entire measurement device 100. The storage device 6 is configured of, for example, a non-volatile semiconductor memory and stores a program executed by the control device 5 and various data used by the control device 5. In addition, a configuration in which functions of the control device 5 are distributed among a plurality of integrated circuits, or a configuration in which some or all of the functions of the control device 5 are realized by a dedicated electronic circuit may also be adopted. Also, although the control device 5 and the storage device 6 are shown as separate elements in FIG. 2, a control device 5 including the storage device 6 can also be realized, for example, by an application specific integrated circuit (ASIC) or the like.

The control device 5 of the embodiment executes the program stored in the storage device 6 to identify the biological information of the subject from the plurality of detection signals S (S1, S2, and S3) generated by the detection device 3. Specifically, the control device 5 can identify a pulse interval (PPI) of the subject from the detection signal S1 indicating the intensity of the green light LG received by the first light receiving part 51. In addition, the control device 5 can identify an oxygen saturation (SpO2) of the subject by analyzing the detection signal S2 indicating the intensity of the red light LR received by the second light receiving part 61 and the detection signal S3 indicating the intensity of the near-infrared light LI received by the second light receiving part 61.

As described above, in the measurement device 100, the control device 5 functions as an information analysis part that identifies the biological information from the detection signals S indicating detection results of the detection device 3. The control device (information analysis part) 5 causes the display device 4 to display the biological information identified from the detection signals S. In addition, it is also possible to notify a user of measurement results by audio output. Further, a configuration in which a warning (possibility of impaired physical function) is notified to the user when a pulse rate or oxygen saturation changes to a value outside of a predetermined range is also suitable.

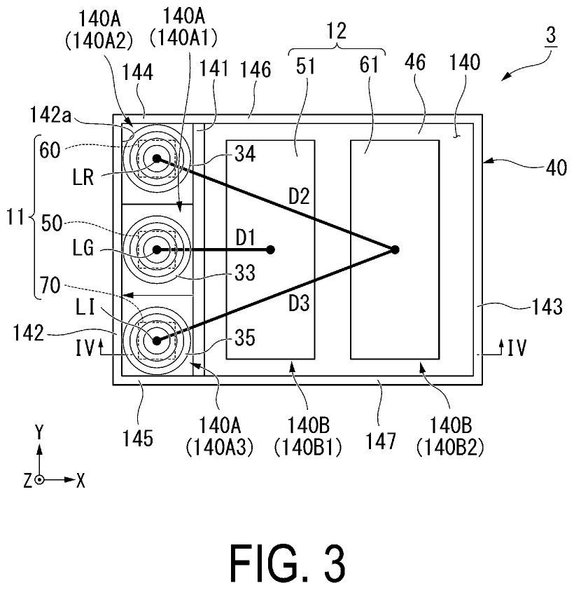

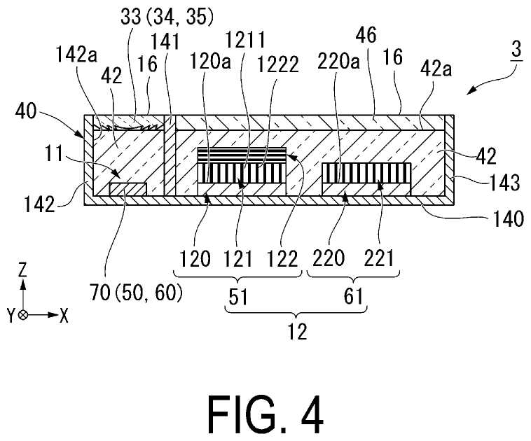

FIG. 3 is a plan view of the detection device 3 of the embodiment. FIG. 4 is a cross-sectional view along line IV-IV in FIG. 3. As shown in FIGS. 3 and 4, the detection device 3 of the embodiment further includes, in addition to the light emitting unit part 11 and the light receiving unit part 12, a case member 40, a encapsulating layer 42, a first optical member 33, a second optical member 34, a third optical member 35, and a cover member 46. Also, in FIGS. 3 and 4, illustration of the drive circuit 13 and the output circuit 14 is omitted.

Arrangements of and positional relations between constituent members of the detection device 3 will be described below using an XYZ coordinate system. An X axis corresponds to an axis extending along a long side (one side) of the case member 40 having a rectangular outer shape, a Y axis corresponds to an axis that is orthogonal to the X axis and extends along a short side (another side) of the case member 40, and a Z axis corresponds to an axis that is orthogonal to the X and Y-axes and extends along a normal line of the detection surface 16 in contact with the measurement site M.

As shown in FIGS. 3 and 4, the case member 40 is a member that holds each element (the first light emitting part 50, the second light emitting part 60, the third light emitting part 70, the first light receiving part 51, and the second light receiving part 61) forming the detection device 3.

The case member 40 is made of, for example, aluminum. Further, any material and manufacturing method are used for the case member 40. For example, the case member 40 can be formed by injection molding of a resin material. In addition, a configuration in which the case member 40 is formed integrally with the housing part 1 is also suitable.

The case member 40 of the embodiment has a bottom plate part 140 having a rectangular flat plate shape, a first wall part 141, a second wall part 142, a third wall part 143, a fourth wall part 144, a fifth wall part 145, a sixth wall part 146, and a seventh wall part 147. Also, at least some of the bottom plate part 140, the first wall part 141, the second wall part 142, the third wall part 143, the fourth wall part 144, the fifth wall part 145, the sixth wall part 146, and the seventh wall part 147 may be integrally formed.

The case member 40 has a first accommodating part 140A and a second accommodating part 140B.

The first accommodating part 140A is defined by the bottom plate part 140, the first wall part 141, the second wall part 142, the fourth wall part 144, and the fifth wall part 145 and accommodates the light emitting unit part 11.

The first accommodating part 140A includes a first light emitting region 140A1 in which the first light emitting part 50 of the light emitting unit part 11 is disposed, a second light emitting region 140A2 in which the second light emitting part 60 of the light emitting unit part 11 is disposed, and a third light emitting region 140A3 in which the third light emitting part 70 of the light emitting unit part 11 is disposed.

The second accommodating part 140B is defined by the bottom plate part 140, the first wall part 141, the third wall part 143, the sixth wall part 146, and the seventh wall part 147 and accommodates the light receiving unit part 12.

The second accommodating part 140B includes a first light receiving region 140B1 in which the first light receiving part 51 of the light receiving unit part 12 is disposed and a second light receiving region 140B2 in which the second light receiving part 61 of the light receiving unit part 12 is disposed.

As shown in FIG. 3, the light emitting parts 50, 60, and 70 are disposed side by side in a first direction along the Y axis at intervals from each other. Specifically, the second light emitting part 60 is disposed on a +Y side of the first light emitting part 50 and the third light emitting part 70 is disposed on a −Y side of the first light emitting part 50. That is, the first light emitting part 50 is disposed between the second light emitting part 60 and the third light emitting part 70 in the direction along the Y axis. Further, it can also be said that the first light emitting part 50 is located between the second light emitting part 60 and the third light emitting part 70.

As shown in FIG. 3, the light receiving parts 51 and 61 are disposed side by side at intervals from each other in the direction along the X axis, which is a second direction intersecting (orthogonal to) the Y axis. Specifically, the first light receiving part 51 is disposed on a +X side of the light emitting unit part 11 and the second light receiving part 61 is disposed on the +X side of the first light receiving part 51. That is, the second light receiving part 61 is disposed on the +X side of the first light receiving part 51 opposite to the light emitting unit part 11.

Here, a distance from the first light emitting part 50 to the first light receiving part 51 is defined as a first distance D1, a distance from the second light emitting part 60 to the second light receiving part 61 is defined as a second distance D2, and a distance from the third light emitting part 70 to the second light receiving part 61 is defined as a third distance D3. The first distance Dl corresponds to a distance between center portions of the first light emitting part 50 and the first light receiving part 51 when viewed, in plan view, in the Z axis direction, which is a third direction intersecting the X axis direction and the Y axis direction. Also, the second distance D2 corresponds to a distance between center portions of the second light emitting part 60 and the second light receiving part 61 when viewed, in plan view, in the Z axis direction. In addition, the third distance D3 corresponds to a distance between center portions of the third light emitting part 70 and the second light receiving part 61 when viewed, in plan view, in the Z axis direction.

In the detection device 3 of the embodiment, in the second direction along the X axis, the first distance Dl from the first light emitting part 50 to the first light receiving part 51 is shorter than the second distance D2 from the second light emitting part 60 to the second light receiving part 61. Also, in the second direction along the X axis, the first distance Dl from the first light emitting part 50 to the first light receiving part 51 is shorter than the third distance D3 from the third light emitting part 70 to the second light receiving part 61. In addition, the second distance D2 and the third distance D3 are equal.

In this way, the detection device 3 of the embodiment adopts a configuration in which the first light receiving part 51 for receiving the green light LG is disposed at a position closest to the first light emitting part 50 that radiates the green light LG.

The first wall part 141 is provided to separate the first accommodating part 140A from the second accommodating part 140B in the direction along the X axis. That is, the first wall part 141 is provided between the light emitting unit part 11 and the light receiving unit part 12. The first wall part 141 is a flat plate-shaped member that protrudes from the bottom plate part 140 to a +Z side and extends in the Y axis direction.

The first wall part 141 has light absorbing properties. The first wall part 141 is formed by, for example, coloring the aluminum material forming the case member 40 in black. Thus, the first wall part 141 functions as a light blocking member that prevents light emitted from the light emitting unit part 11 from being directly incident on the second accommodating part 140B side.

The second wall part 142 is a flat plate-shaped member that protrudes from an peripheral edge of the bottom plate part 140 on a −X side to the +Z side and extends in the Y axis direction. The second wall part 142 is provided on the bottom plate part 140 to face the first wall part 141 in the direction along the X axis. The second wall part 142 is provided on the −X side of the light emitting unit part 11. That is, the second wall part 142 is provided on a side of the light emitting unit part 11 opposite to the light receiving unit part 12. The first wall part 141 and the second wall part 142 are disposed side by side in the direction along the X axis with an interval therebetween.

The second wall part 142 has light reflecting properties. In the case of the embodiment, the second wall part 142 includes a reflection surface 142a provided on its inner surface side facing the light emitting unit part 11. The reflection surface 142a is formed by, for example, providing a mirror film on a surface of the aluminum material forming the case member 40. Also, the reflecting surface 142a may be configured of a polished surface obtained by polishing the aluminum material.

The third wall part 143 is a flat plate-shaped member that protrudes from an peripheral edge of the bottom plate part 140 on the +X side to the +Z side and extends in the Y axis direction. The third wall part 143 is provided on the bottom plate part 140 to face the first wall part 141 in the direction along the X axis.

The fourth wall part 144 is provided on the +Y side of the first wall part 141 and the second wall part 142. The fourth wall part 144 is a flat plate-shaped member that protrudes from an peripheral edge of the bottom plate part 140 on the +Y side to the +Z side and extends in the X axis direction.

The fifth wall part 145 is provided on the −Y side of the first wall part 141 and the second wall part 142. The fifth wall part 145 is a flat plate-shaped member that protrudes from an peripheral edge of the bottom plate part 140 on the −Y side to the +Z side and extends in the X axis direction. The fifth wall part 145 is provided on the bottom plate part 140 to face the fourth wall part 144 in the direction along the Y axis.

In the case of the embodiment, the fourth wall part 144 and the fifth wall part 145 have light reflecting properties similarly to the second wall part 142. Thus, light radiated from the light emitting unit part 11 in the Y direction is reflected by the fourth wall part 144 and the fifth wall part 145 and is incident on the living body, and thus light use efficiency of the light emitting unit part 11 can be improved.

The sixth wall part 146 is a flat plate-shaped member that protrudes from an peripheral edge of the bottom plate part 140 on the +Y side to the +Z side and extends in the X axis direction. The seventh wall part 147 is a flat plate-shaped member that protrudes from an peripheral edge of the bottom plate part 140 on the −Y side to the +Z side and extends in the X axis direction. The seventh wall part 147 is provided on the bottom plate part 140 to face the sixth wall part 146 in the direction along the Y axis.

In the case of the embodiment, the third wall part 143, the sixth wall part 146, and the seventh wall part 147 have light absorbing properties similarly to the first wall part 141. Thus, incidence of the light reflected by the third wall part 143, the sixth wall part 146, and the seventh wall part 147 on the light receiving unit part 12 as stray light can be inhibited.

The encapsulating layer 42 is made of a light transmitting resin material filled in a gap between the case member 40 and the light emitting unit part 11 and the light receiving unit part 12 that are accommodated in the case member 40. The encapsulating layer 42 encapsulates (molds) the light emitting unit part 11 and the light receiving unit part 12 in the case member 40.

In the case of the embodiment, an upper surface of the encapsulating layer 42 is provided to be lower than an upper surface of the case member 40. Specifically, each of upper parts of the first to seventh wall parts 141 to 147 protrudes from the upper surface 42a of the encapsulating layer 42.

Light emitting surfaces of the light emitting parts 50, 60, and 70 are installed in the case member 40 to be parallel to an XY plane. That is, the light emitting parts 50, 60, and 70 are configured to emit light toward the +Z side.

The cover member 46 is provided on the encapsulating layer 42 that covers the second accommodating part 140B.
when viewed, in plan view, in the direction along the Z axis, the cover member 46 is disposed on the encapsulating layer 42 by being fitted into a region surrounded by the first wall part 141, the third wall part 143, the sixth wall part 146, and the seventh wall part 147. The cover member 46 is made of a translucent member. A surface of the cover member 46 on a side opposite to the encapsulating layer 42 forms a part of the detection surface 16.

The first optical member 33, the second optical member 34, and the third optical member 35 are provided on the encapsulating layer 42 that covers the first accommodating part 140A. Surfaces of the first optical member 33, the second optical member 34, and the third optical member 35 on a side opposite to the encapsulating layer 42 are flat surfaces and form a part of the detection surface 16.

The first optical member 33 refracts the green light (first light) LG, the second optical member 34 refracts the red light (second light) LR, and the third optical member 35 refracts the near-infrared light (third light) LR. Specifically, the first optical member 33, the second optical member 34, and the third optical member 35 are configured by Fresnel lenses in which curved lens surfaces are replaced with concentric grooves. Since the first optical member 33, the second optical member 34, and the third optical member 35 have plate shapes, they also function as covers that cover the encapsulating layer 42 together with the cover member 46.

In the case of the embodiment, thicknesses of the first optical member 33, the second optical member 34, and the third optical member 35 configured by the Fresnel lenses are curbed, and thus a thickness of the cover that covers the encapsulating layer 42 can be curbed. Thus, the detection device 3 of the embodiment can improve detection accuracy of the light receiving unit part 12 by bringing the light emitting unit part 11 and the light receiving unit part 12 accommodated in the case member 40 close to the living body.
when viewed, in plan view, in the direction along the Z axis, the first optical member 33 is disposed at a position at which it overlaps at least a part of the first light emitting region 140A1 of the case member 40, the second optical member 34 is disposed at a position at which it overlaps at least a part of the second light emitting region 140A2 of the case member 40, and the third optical member 35 is disposed at a position at which it overlaps at least a part of the third light emitting region 140A3 of the case member 40. In the case of the embodiment, the first optical member 33 is disposed to overlap the entire region of the first light emitting part 50, the second optical member 34 is disposed to overlap the entire region of the second light emitting part 60, and the third optical member 35 is disposed to overlap the entire region of the third light emitting part 70. For this reason, the first optical member 33, the second optical member 34, and the third optical member 35 can efficiently swallow light emitted from the corresponding light emitting parts 50, 60, and 70.

In the case of the embodiment, the first optical member 33 is disposed such that a center of the concentric grooves replacing the curved lens surface substantially coincides with a center of the first light emitting part 50. Similarly to the first optical member 33, the second optical member 34 is disposed such that a center of the concentric grooves substantially coincide with a center of the second light emitting part 60, and the third optical member 35 is disposed such that a center of the concentric grooves substantially coincides with a center of the third light emitting part 70.

That is, the first optical member 33 is provided to correspond to the first light emitting part 50, the second optical member 34 is provided to correspond to the second light emitting part 60, and the third optical member 35 is provided to correspond to the third light emitting part 70.
when viewed, in plan view, in the direction along the Z axis, the first optical member 33, the second optical member 34, and the third optical member 35 are disposed on the encapsulating layer 42 by being fitted into a region surrounded by the first wall part 141, the second wall part 142, the fourth wall part 144, and the fifth wall part 145. The first optical member 33, the second optical member 34, and the third optical member 35 are disposed with the concentric grooves in contact with the encapsulating layer 42. An air layer is interposed in a gap between the first optical member 33, the second optical member 34, and the third optical member 35 and the encapsulating layer 42.

The first optical member 33, the second optical member 34, and the third optical member 35 are held between the first wall part 141 and the second wall part 142 in the second direction along the X axis. In addition, the first optical member 33, the second optical member 34, and the third optical member 35 are held between the fourth wall part 144 and the fifth wall part 145 in the first direction along the Y axis.

For this reason, each of the first optical member 33, the second optical member 34, and the third optical member 35 is accurately positioned on the first light emitting part 50, the second light emitting part 60, and the third light emitting part 70 in the directions along the X axis and the Y axis.

A focal length of the first optical member 33 is designed to optimize directivity of the green light LG incident thereon from the first light emitting part 50. The first optical member 33 condenses the green light LG incident thereon from the first light emitting part 50 and causes it to be efficiently incident on the living body.

A focal length of the second optical member 34 is designed to optimize directivity of the red light LR incident thereon from the second light emitting part 60. The second optical member 34 condenses the red light LR incident thereon from the second light emitting part 60 and causes it to be efficiently incident on the living body.

A focal length of the third optical member 35 is designed to optimize directivity of the near-infrared light LI incident thereon from the third light emitting part 70. The third optical member 35 condenses the near-infrared light LI incident thereon from the third light emitting part 70 and causes it to be efficiently incident on the living body.

In the case of the embodiment, the focal length of the first optical member 33 is shorter than the focal length of the second optical member 34. That is, the focal length of the second optical member 34 is longer than the focal length of the first optical member 33. The focal length of the third optical member 35 is longer than the focal length of the first optical member 33. In the case of the embodiment, the focal length of the third optical member 35 is set to be substantially the same as the focal length of the second optical member 34.

As shown in FIG. 4, the first light receiving part 51 includes a sensor 120, an angle limiting filter 121, and a bandpass filter (optical filter) 122.

The sensor 120 is configured of, for example, a photodiode (PD). The angle limiting filter 121 is provided to cover the entire light receiving surface 120a of the sensor 120. The angle limiting filter 121 is formed, for example, by embedding a plug 1222 made of a light blocking material such as tungsten in a silicon oxide layer 1211 having optical transparency.

The silicon oxide layer 1211 forms an optical path for guiding light to the light receiving surface 120a of the sensor 120. The plug 1222 embedded in the silicon oxide layer 1211 limits an incident angle of light passing through the optical path (silicon oxide layer 1211). That is, when the light incident on the silicon oxide layer 1211 is inclined by more than a predetermined angle with respect to the optical path, the incident light hits the plug 1222, some of the light is absorbed by the plug 1222, and the rest is reflected. In addition, since an intensity of the reflected light weakens due to repeated reflections while the light passes through the optical path, the light that can finally pass through the angle limiting filter 121 is substantially limited to the light whose inclination with respect to the optical path is within a predetermined limiting angle.

The angle limiting filter 121 has a characteristic of transmitting light incident thereon at an angle smaller than a predetermined incident angle and blocking light incident thereon at an angle greater than the predetermined incident angle without transmitting the light. Thus, the angle limiting filter 121 can limit an incident angle of light incident on the sensor 120. Specifically, the angle limiting filter 121 transmits the light incident thereon at a predetermined incident angle (hereinafter referred to as an allowable incident angle) due to having propagated in the living body and blocks the light incident thereon at an angle greater than the allowable incident angle such as external light such as sunlight or light that has not entered the living body.

The bandpass filter 122 has a characteristic of selectively transmitting the wavelength band of the green light LG and absorbing and blocking the red light LR and the near-infrared light LI, which are light having other wavelength bands. The bandpass filter 122 is formed, for example, by alternately laminating a plurality of low refractive index layers made of silicon oxide and high refractive index layers made of titanium oxide on the angle limiting filter 121.

Further, the second light receiving part 61 includes a sensor 220 that receives the red light LR or the near-infrared light LI and an angle limiting filter 221 that limits an incident angle of the red light LR or the near-infrared light LI that reaches the sensor 220. That is, in the detection device 3 of the embodiment, the second light receiving part 61 has a different configuration from the first light receiving part 51 in that it does not include a bandpass filter that selectively transmits the red light LR or the near-infrared light LI.

The sensor 220 is configured of, for example, a photodiode. The angle limiting filter 221 is provided on the light receiving surface 220a of the sensor 220. The angle limiting filter 221 has the same configuration as the angle limiting filter 121 and can limit the incident angle of the red light LR or the near-infrared light LI that reaches the sensor 220. For example, the angle limiting filter 221 transmits the red light LR or the near-infrared light LI that propagates in the living body and is incident thereon at an allowable incident angle and blocks the light incident thereon at an angle greater than the allowable incident angle such as external light such as sunlight, or the red light LR or the near-infrared light LI that has not passed through the living body.

Here, some of the red light LR radiated from the second light emitting part 60 or the near-infrared light LI radiated from the third light emitting part 70 may pass through the living body and be incident on the first light receiving part 51. In the case of the embodiment, since the first light receiving part 51 includes the bandpass filter 122 that selectively transmits the green light LG, it is possible to block the red light LR and the near-infrared light LI that have different wavelength bands from the green light LG. Accordingly, the first light receiving part 51 can efficiently receive the green light LG radiated from the first light emitting part 50.

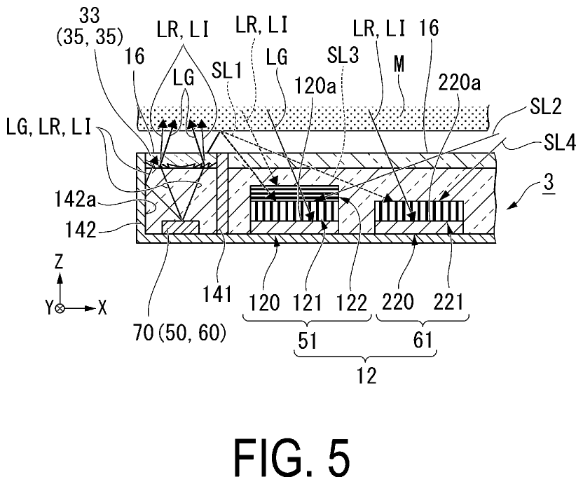

Operations of the detection device 3 of the embodiment will be described below. FIG. 5 is a diagram for describing an operation of the detection device 3.

In the detection device 3 of the embodiment, the green light LG radiated from the first light emitting part 50 is radiated in all directions. As shown in FIG. 5, some of the green light LG radiated from the first light emitting part 50 to the +X side is blocked by the first wall part 141 having light absorption properties. Thus, it is possible to inhibit a decrease in detection accuracy due to some of the green light LG radiated from the first light emitting part 50 being directly incident on the light receiving unit part 12.

In the detection device 3 of the embodiment, some of the green light LG radiated to the −X side is incident on the second wall part 142 having light reflecting properties. The green light LG incident on the second wall part 142 is reflected by the reflection surface 142a and is efficiently incident on the living body via the first optical member 33.

Similarly, some of the red light LR or the near-infrared light LI radiated to the −X side is reflected by the second wall part 142 having light reflecting properties and is incident on the living body via the second optical member 34 and the third optical member 35.

Also, in a region not shown, some of the red light LR radiated to the +Y side from the second light emitting part 60 disposed near the fourth wall part 144 is reflected by the fourth wall part 144 having light reflecting properties and is incident on the living body via the second optical member 34. In addition, some of the near-infrared light LI radiated to the −Y side from the third light emitting part 70 disposed near the fifth wall part 145 is reflected by the fifth wall part 145 having light reflecting properties and is incident on the living body via the third optical member 35. Thus, light use efficiency of the light emitting unit part 11 can be improved.

The present inventors have found that green light is more easily attenuated in a living body than red light or near-infrared light and cannot propagate for a long time in a living body. On the other hand, in the detection device 3 of the embodiment, the first distance D1 between the first light emitting part 50 and the first light receiving part 51 is made smaller than the distance between the second light emitting part 60 or the third light emitting part 70 and the second light receiving part 61 (the second distance D2 or the third distance D3).

In the case of the embodiment, since the first light receiving part 51 is disposed at a position closest to the first light emitting part 50, the green light LG that propagates in the living body and is incident on the first light receiving part 51 can be efficiently received. In addition, as described above, the red light LR and the near-infrared light LI can propagate farther in the living body than the green light LG. For that reason, the second light receiving part 61 can efficiently receive the red light LR and the near-infrared light LI that have propagated longer distances in the living body than the green light LG.

In this way, the detection device 3 of the embodiment can accurately detect the green light LG, the red light LR, and the near-infrared light LI that have propagated in the living body using the light receiving unit part 12.

Further, as shown in FIG. 5, in the detection device 3 of the embodiment, some of the green light LG radiated from the first light emitting part 50 may be reflected, for example, on the surface of the living body (measurement site M) and be directly incident on the first light receiving part 51 without passing through the living body. Also, external light such as sunlight may be directly incident on the first light receiving part 51 through a gap between the living body and the detection surface 16. Hereinafter, green light LG that goes toward the first light receiving part 51 without passing through the living body will be referred to as a “first stray light component SL1,” and external light that goes directly toward the first light receiving part 51 will be referred to as a “second stray light component SL2.”

Since the first stray light component SL1 has a green wavelength band, it passes through the bandpass filter 122 and is incident on the angle limiting filter 121 provided in a layer below the bandpass filter 122. As described above, the angle limiting filter 121 has a characteristic of transmitting light incident thereon at an angle smaller than the allowable incident angle and blocking light incident thereon at an angle greater than the allowable incident angle.

Since the first stray light component SL1 is incident on the first light receiving part 51 without passing through the living body, an incident angle of the green light LG with respect to the first light receiving part 51 is greater than the allowable incident angle of the angle limiting filter 121. That is, the first stray light component SL1 is blocked by the angle limiting filter 121. Thus, the first light receiving part 51 can inhibit incidence of the first stray light component SL1 on the light receiving surface 120a of the sensor 120 using the angle limiting filter 121.

The second stray light component SL2 is mostly blocked by the bandpass filter 122, but a component having a green wavelength band included in the second stray light component SL2 is transmitted through the bandpass filter 122. Here, as described above, since the second stray light component SL2 is incident through the gap between the living body and the detection surface 16, an incident angle of the second stray light component SL2 with respect to the first light receiving part 51 is greater than the allowable incident angle of the angle limiting filter 121. For that reason, some of the second stray light component SL2 (the component having a green wavelength band) transmitted through the bandpass filter 122 is blocked by the angle limiting filter 121. Thus, the first light receiving part 51 can inhibit the incidence of the second stray light component SL2 on the light receiving surface 120a of the sensor 120 using the angle limiting filter 121.

As described above, in the detection device 3 of the embodiment, the green light LG radiated from the light emitting unit part 11 and passing through the living body can be efficiently incident on the light receiving surface 120a of the sensor 120. In addition, the detection device 3 of the embodiment can make it difficult for the first stray light component SL1 and the second stray light component SL2 to be incident on the light receiving surface 120a of the sensor 120.

Accordingly, the first light receiving part 51 can obtain a high S/N ratio by inhibiting the incidence of the first stray light component SL1 and the second stray light component SL2, which are noise components. For that reason, the detection device 3 of the embodiment can receive the green light LG with high accuracy in the first light receiving part 51, and thus by inhibiting an amount of light emission of the green light LG in the first light emitting part 50, power consumption of the light emitting unit part 11 can be inhibited.

In the detection device 3 of the embodiment, the focal length of the first optical member 33 is shorter than the focal length of the second optical member 34. For this reason, the first optical member 33 can cause the green light LG to be incident on a position close to a surface layer in the living body. The green light LG incident near the surface layer in the living body has a shorter propagation distance in the living body. The green light LG radiated from the living body is efficiently incident on the first light receiving part 51 disposed near the first light emitting part 50 in the direction along the X axis. Accordingly, the first light receiving part 51 can efficiently receive the green light LG propagated in and radiated from the living body.

In addition, in the detection device 3 according to the embodiment, the distance between the second light emitting part 60 or the third light emitting part 70 and the second light receiving part 61 (the second distance D2 or the third distance D3) is greater than the first distance Dl between the first light emitting part 50 and the first light receiving part 51. That is, distances that the red light LR and the near-infrared light LI propagate in the living body until they are incident on the second light receiving part 61 are longer than a distance that the green light LG propagates in the living body until it is incident on the first light receiving part 51.

As described above, the green light LG can only propagate a short distance in the living body than the red light LR or the near-infrared light LI. For that reason, even if some of the green light LG propagates in the living body until it can reach the second light receiving part 61, the green light LG will be in a sufficiently attenuated state when it passes through the living body. Accordingly, the green light LG cannot be incident on the second light receiving part 61.

On the other hand, in the detection device 3 of the embodiment, the focal length of the second optical member 34 is longer than the focal length of the first optical member 33, and the focal length of the third optical member 35 is longer than the focal length of the first optical member 33. For this reason, the second optical member 34 or the third optical member 35 can allow the red light LR or the near-infrared light LI to enter deep into the living body. The red light LR and the near-infrared light LI that have entered deep into the living body have longer propagation distances in the living body. The red light LR and the near-infrared light LI can propagate farther in the living body than the green light LG. For that reason, even when the red light LR and the near-infrared light LI have propagated longer distances in the living body than the green light LG, they are in a state of having a sufficient amount of light for the second light receiving part 61 located further away from the light emitting unit part 11. Accordingly, the red light LR and the near-infrared light LI radiated from the living body are efficiently incident on the second light receiving part 61 disposed farther than the first light receiving part 51 in the direction along the X axis. Accordingly, the second light receiving part 61 can efficiently receive the red light LR and the near-infrared light LI propagated in the living body and radiated.

In the case of the embodiment, since only the red light LR and the near-infrared light LI are incident on the second light receiving part 61, a bandpass filter that selectively transmits the red light LR and the near-infrared light LI and blocks the green light LG is not provided in the second light receiving part 61. That is, the detection device 3 of the embodiment can adopt the configuration in which only the first light receiving part 51 includes the bandpass filter 122 while the second light receiving part 61 does not include a bandpass filter. Accordingly, the detection device 3 of the embodiment can reduce costs by omitting the bandpass filter of the second light receiving part 61.

In addition, some of the red light LR radiated from the second light emitting part 60 or some of the near-infrared light LI radiated from the third light emitting part 70 may be directly incident on the second light receiving part 61 without passing through the living body. Also, external light such as sunlight may be directly incident on the second light receiving part 61 through the gap between the living body and the detection surface 16. Hereinafter, the red light LR or the near-infrared light LI directed directly to the second light receiving part 61 without passing through the living body will be collectively referred to as a “third stray light component SL3” and external light directed directly to the second light receiving part 61 will be referred to as a “fourth stray light component SL4.”

Since the third stray light component SL3 is incident on the angle limiting filter 221 without passing through the living body, the incident angle of the third stray light component SL3 with respect to the second light receiving part 61 is greater than the allowable incident angle of the angle limiting filter 221. Further, since the fourth stray light component SL4 is incident through the gap between the living body and the detection surface 16, the incident angle of the fourth stray light component SL4 with respect to the second light receiving part 61 is greater than the allowable incident angle of the angle limiting filter 221.

For that reason, the third stray light component SL3 and the fourth stray light component SL4 are satisfactorily blocked by the angle limiting filter 221. Thus, the second light receiving part 61 can inhibit incidence of the third stray light component SL3 and the fourth stray light component SL4 on the light receiving surface 220a of the sensor 220 using the angle limiting filter 221.

As described above, in the detection device 3 of the embodiment, the red light LR or the near-infrared light LI radiated from the light emitting unit part 11 and passing through the living body can be efficiently incident on the light receiving surface 220a of the sensor 220. In addition, in the detection device 3 of the embodiment, it is possible to make it difficult for the third stray light component SL3 and the fourth stray light component SL4 to be incident on the light receiving surface 220a of the sensor 220.

Accordingly, the second light receiving part 61 can obtain a high S/N ratio by inhibiting the incidence of the third stray light component SL3 and the fourth stray light component SL4, which are noise components. According to the detection device 3 of the embodiment, since the red light LR and the near-infrared light LI are efficiently received in the second light receiving part 61, it is possible to inhibit each amount of light emitted by the second light emitting part 60 and the third light emitting part 70, thereby inhibiting the power consumption of the light emitting unit part 11.

The detection device 3 of the embodiment includes the first light emitting part 50 that emits the green light LG having the green wavelength band toward the living body, the second light emitting part 60 that emits the red light LR having the red wavelength band longer than the green wavelength band toward the living body, the third light emitting part 70 that emits the near-infrared light LI having the near-infrared wavelength band longer than the red wavelength band toward the living body, the first light receiving part 51 that receives the green light LG emitted from the first light emitting part 50 and radiated from the living body, the second light receiving part 61 that receives the red light LR and the near-infrared light LI emitted from the second light emitting part 60 and the third light emitting part 70 and radiated from the living body, the first optical member 33 that refracts the green light LG, the second optical member 34 that refracts the red light LR, and the third optical member 35 that refracts the near-infrared light LI.

Since the detection device 3 of the embodiment includes the first optical member 33, the second optical member 34, and the third optical member 35 individually provided to correspond to the first light emitting part 50, the second light emitting part 60, and the third light emitting part 70, it is possible to individually optimize the directivity of the green light LG, the red light LR, and the near-infrared light LI. Thus, the first light receiving part 51 and the second light receiving part 61 can detect the green light LG, the red light LR, or the near-infrared light LI that have passed through the living body with high accuracy. Accordingly, it is possible to provide a highly reliable detection device having high light use efficiency of the light emitting parts 50, 60, and 70 and high detection accuracy.

In addition, according to the measurement device 100 of this embodiment, since the measurement device 100 includes the above detection device 3, it is possible to provide a biometric device that enables highly accurate detection while reducing power consumption.

### Second Embodiment

Next, a detection device of a second embodiment will be described. The detection device of the embodiment is different from that of the first embodiment in the configurations of the first optical member, the second optical member, and the third optical member. Hereinafter, configurations and members common to those in the first embodiment will be denoted by the same reference numerals, and reference numerals for details will be omitted.

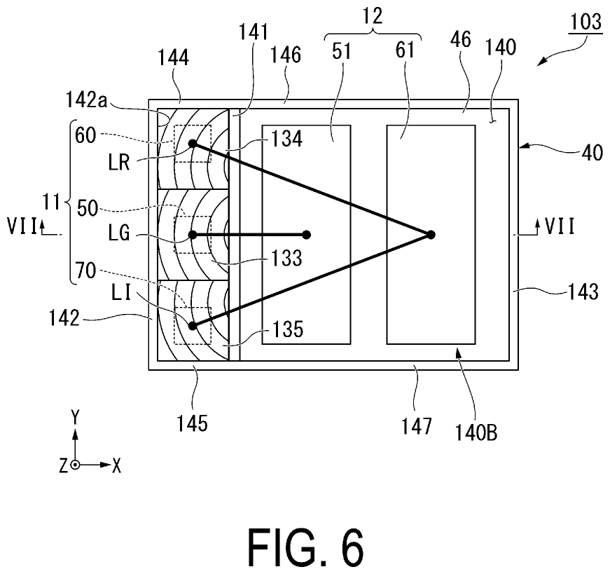

FIG. 6 is a plan view showing a main part configuration of a detection device 103 of the embodiment.

As shown in FIG. 6, the detection device 103 of the embodiment further includes the light emitting unit part 11, the light receiving unit part 12, the case member 40, the encapsulating layer 42, a first optical member 133, a second optical member 134, a third optical member 135, and the cover member 46.

The first optical member 133, the second optical member 134, and the third optical member 135 are configured of eccentric Fresnel lenses.

Specifically, the first optical member 133 has a structure in which a center of concentric grooves replacing a curved lens surface is shifted to the first light receiving part 51 side with respect to the center of the first light emitting part 50 on a virtual line connecting the centers of the first light emitting part 50 and the first light receiving part 51 to each other. The second optical member 134 has a configuration in which a center of concentric grooves replacing a curved lens surface is shifted to the second light receiving part 61 side with respect to the center of the second light emitting part 60 on a virtual line connecting the centers of the second light emitting part 60 and the second light receiving part 61 to each other. The third optical member 135 has a configuration in which a center of concentric grooves replacing a curved lens surface is shifted to the second light receiving part 61 side with respect to the center of the third light emitting part 70 on a virtual line connecting the centers of the third light emitting part 70 and the second light receiving part 61 to each other.

That is, in the embodiment, center positions of the first light emitting part 50 and the first optical member 33 are different from each other, center positions of the second light emitting part 60 and the second optical member 34 are different from each other, and center positions of the third light emitting part 70 and the third optical member 35 are different from each other.

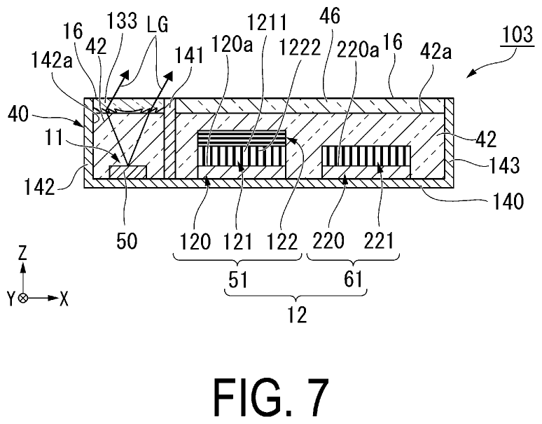

Operations of the detection device 103 of the embodiment will be described below. FIG. 7 is a cross-sectional view along line VII-VII in FIG. 6.

As shown in FIG. 6, the green light LG radiated from the first light emitting part 50 is refracted toward the first light receiving part 51 side by the first optical member 133 configured of an eccentric Fresnel lens and is incident on the living body from an oblique direction.

Also, although not shown, the red light LR radiated from the second light emitting part 60 is refracted toward the second light receiving part 61 side by the second optical member 134 configured of an eccentric Fresnel lens and is incident on the living body from an oblique direction.

Also, although not shown, the near-infrared light LI radiated from the third light emitting part 70 is refracted toward the second light receiving part 61 side by the third optical member 135 configured of an eccentric Fresnel lens and is incident on the living body from an oblique direction.

In this way, according to the detection device 103 of the embodiment, by controlling the directivity of the light radiated from the light emitting parts 50, 60, and 70, it is possible to inhibit an amount of light diffused in directions opposite to the light receiving parts 51 and 61. Accordingly, since the light radiated from each of the light emitting parts 50, 60, and 70 propagates through the living body and is efficiently incident on the light receiving parts 51 and 61, it is possible to provide a detection device having high light use efficiency and high light reception accuracy.

Further, in the embodiment, a case in which the center positions of the light emitting parts and the optical members are different from each other has been described as an example, but at least one thereof may have a different configuration.

### Third Embodiment

Next, a detection device of a third embodiment will be described. The detection device of the embodiment is different from that of the first embodiment in the configurations of the first optical member, the second optical member, and the third optical member. Hereinafter, configurations and members common to those in the first embodiment will be denoted by the same reference numerals, and reference numerals for details will be omitted.

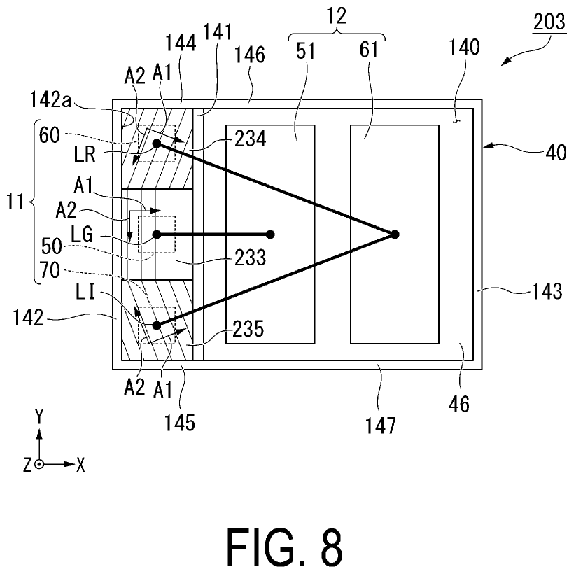

FIG. 8 is a plan view showing a main part configuration of a detection device 203 of the embodiment.

As shown in FIG. 8, the detection device 203 of the embodiment further includes the light emitting unit part 11, the light receiving unit part 12, the case member 40, the encapsulating layer 42, a first optical member 233, a second optical member 234, a third optical member 235, and the cover member 46.

The first optical member 233, the second optical member 234, and the third optical member 235 of the embodiment are configured of linear Fresnel lenses. The linear Fresnel lens has a cross-section in a first direction A1 having a stepped shape similar to the Fresnel lens of the above-described embodiment and a cross-section in a second direction A2 orthogonal to the first direction A1 has a planar shape. That is, the linear Fresnel lens has a condensing function as a lens in the first direction A1, but does not have a condensing function and transmits light in the second direction A2.
when viewed, in plan view, in the direction along the Z axis, the first optical member 233, the second optical member 234, and the third optical member 235 are disposed on the encapsulating layer 42 by being fitted into the region surrounded by the first wall part 141, the second wall part 142, the fourth wall part 144, and the fifth wall part 145.

In the embodiment, the first optical member 233 is configured so that the first direction A1 having a lens function can be disposed along a virtual line connecting centers of the first optical member 233 and the first light receiving part 51 to each other. The second optical member 234 is configured so that the first direction A1 having a lens function can be disposed along a virtual line connecting centers of the second optical member 234 and the second light receiving part 61 to each other. The third optical member 235 is configured so that the first direction A1 having a lens function can be disposed along a virtual line connecting centers of the third optical member 235 and the second light receiving part 61 to each other.

In the detection device 203 of the embodiment, the green light LG radiated from the first light emitting part 50 is refracted toward the first light receiving part 51 side by the first optical member 233 configured of a linear Fresnel lens and is incident on the living body from an oblique direction. Also, the red light LR radiated from the second light emitting part 60 is refracted toward the second light receiving part 61 side by the second optical member 234 configured of a linear Fresnel lens and is incident on the living body from an oblique direction. Also, the near-infrared light LI radiated from the third light emitting part 70 is refracted toward the second light receiving part 61 by the third optical member 235 configured of a linear Fresnel lens and is incident on the living body from an oblique direction.

In this way, according to the detection device 203 of the embodiment, by controlling the directivity of the light radiated from the light emitting parts 50, 60, and 70, it is possible to inhibit the amount of light diffused in the directions opposite to the light receiving parts 51 and 61. Accordingly, since the light radiated from the light emitting parts 50, 60, and 70 propagates in the living body and is efficiently incident on the light receiving parts 51 and 61, it is possible to provide a detection device having high light use efficiency and high light reception accuracy.

### Fourth Embodiment

Next, a detection device of a fourth embodiment will be described. The detection device of the embodiment is different from that of the first embodiment in that optical members are provided on the light receiving part side. Hereinafter, configurations and members common to those in the first embodiment will be denoted by the same reference numerals, and reference numerals for details will be omitted.

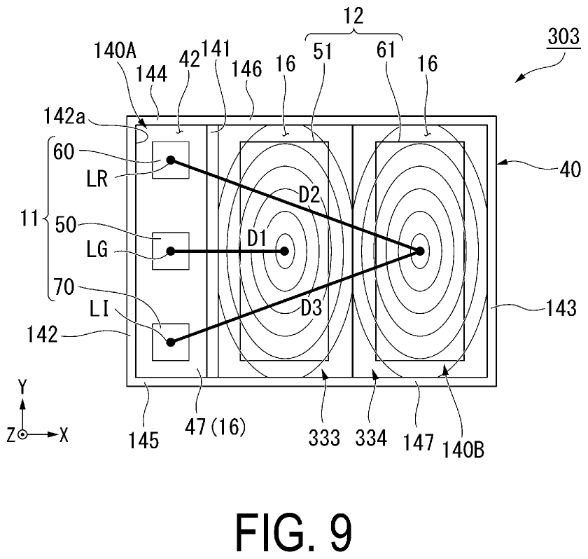

FIG. 9 is a plan view showing a main part configuration of a detection device 303 of the embodiment.

As shown in FIG. 9, the detection device 303 of the embodiment further includes the light emitting unit part 11, the light receiving unit part 12, the case member 40, the encapsulating layer 42, a first optical member 333, a second optical member 334, and a cover member 47.

In the embodiment, the cover member 47 is provided on the encapsulating layer 42 that covers the first accommodating part 140A.
when viewed, in plan view, in the direction along the Z axis, the cover member 47 is disposed on the encapsulating layer 42 by being fitted into the region surrounded by the first wall part 141, the second wall part 142, the fourth wall part 144, and the fifth wall part 145. The cover member 47 is made of a translucent member. A surface of the cover member 47 on a side opposite to the encapsulating layer 42 forms a part of the detection surface 16.

The first optical member 333 and the second optical member 334 are provided on the encapsulating layer 42 that covers the second accommodating part 140B. Surfaces of the first optical member 333 and the second optical member 334 on a side opposite to the encapsulating layer 42 are flat and form a part of the detection surface 16.

The first optical member 333 and the second optical member 334 are configured of Fresnel lenses in which a curved lens surface is replaced with grooves in which ellipses are concentrically arranged. Since the first optical member 333 and the second optical member 334 have plate shapes, they also function as a cover that covers the encapsulating layer 42 together with the cover member 47.

In the case of the embodiment, since the thicknesses of the first optical member 333, the second optical member 334, and the third optical member 335 configured by the Fresnel lenses are curbed, the thickness of the cover that covers the encapsulating layer 42 can be curbed. Thus, the detection device 3 of the embodiment can improve the detection accuracy of the light receiving unit part 12 by bringing the light emitting unit part 11 and the light receiving unit part 12 accommodated in the case member 40 close to the living body.
when viewed, in plan view, in the direction along the Z axis, the first optical member 333 is disposed at a position at which it overlaps at least a part of the first light receiving part 51, and the second optical member 334 is disposed at a position at which it overlaps at least a part of the second light receiving part 61. In the case of the embodiment, the first optical member 333 is configured of a Fresnel lens having grooves in which ellipses are concentrically disposed, and thus is disposed to overlap the entire region of the rectangular first light receiving part 51 while inhibiting enlargement of its lens shape. In addition, the second optical member 334 is configured of a Fresnel lens having grooves in which ellipses are concentrically arranged, and thus is disposed to overlap the entire region of the second light receiving part 61 while inhibiting enlargement of its lens shape.

In the embodiment, the first optical member 333 is disposed such that a center of concentric grooves substantially coincides with the center of the first light receiving part 51. Similarly to the first optical member 333, the second optical member 334 is disposed such that a center of concentric grooves substantially coincides with the center of the second light receiving part 61.

That is, the first optical member 333 is provided to correspond to the first light receiving part 51, and the second optical member 334 is provided to correspond to the second light receiving part 61.
when viewed, in plan view, in the direction along the Z axis, the first optical member 333 and the second optical member 334 are disposed on the encapsulating layer 42 by being fitted into the region surrounded by the first wall part 141, the third wall part 143, the sixth wall part 146, and the seventh wall part 147. The first optical member 333 and the second optical member 334 are disposed with the concentric grooves in contact with the encapsulating layer 42. An air layer is interposed in a gap between the encapsulating layer 42, the first optical member 333, and the second optical member 334.

The first optical member 333 and the second optical member 334 are held between the first wall part 141 and the third wall part 143 in the second direction along the X axis. In addition, the first optical member 333 and the second optical member 334 are held between the sixth wall part 146 and the seventh wall part 147 in the first direction along the Y axis.

For this reason, each of the first optical member 333 and the second optical member 334 is positioned on the first light receiving part 51 and the second light receiving part 61 with high accuracy in the directions along the X axis and the Y axis.
when viewed, in plan view, in the direction along the Z axis, an area of the first optical member 333 is greater than an area of the first light receiving part 51, and an area of the second optical member 334 is greater than an area of the second light receiving part 61.

A focal length of the first optical member 333 is designed such that the green light LG, which is incident thereon from the first light emitting part 50, propagates through the living body, and is radiated, is condensed and efficiently captured by the first light receiving part 51. For this reason, the first optical member 333 condenses the green light LG incident thereon from the living body with a width wider than that of the first light receiving part 51 and causes it to be efficiently incident on the first light receiving part 51. Accordingly, even when a size of the first light receiving part 51 is reduced, the first optical member 333 can condense the green light LG and cause it to be incident on the first light receiving part 51.

A focal length of the second optical member 334 is designed such that the red light LR or the near-infrared light LI, which is incident thereon from the second light emitting part 60, propagates in the living body, and is radiated, is condensed and efficiently captured by the second light receiving part 61. For this reason, the second optical member 334 condenses the red light LR or the near-infrared light LI incident thereon from the living body with a width wider than that of the second light receiving part 61 and causes it to be efficiently incident on the second light receiving part 61. Accordingly, even when a size of the second light receiving part 61 is reduced, the second optical member 334 can condense the red light LR or the near-infrared light LI and cause it to be incident on the second light receiving part 61.

As described above, since the detection device 303 of the embodiment includes the first optical member 333 and the second optical member 334 individually provided to correspond to the first light receiving part 51 and the second light receiving part 61, it is possible to optimize the directivity of the green light LG, the red light LR, and the near-infrared light LI. Thus, the first light receiving part 51 and the second light receiving part 61 can detect the green light LG, the red light LR, or the near-infrared light LI that have passed through the living body with high accuracy. Accordingly, it is possible to provide a highly reliable detection device having high light use efficiency of the light emitting parts 50, 60, and 70 and high detection accuracy.

### Fifth Embodiment

Next, a detection device of a fifth embodiment will be described. The detection device of the embodiment is different from the fourth embodiment in the configurations of the first optical member and the second optical member. Hereinafter, configurations and members common to those in the fourth embodiment will be denoted by the same reference numerals, and reference numerals for details will be omitted.

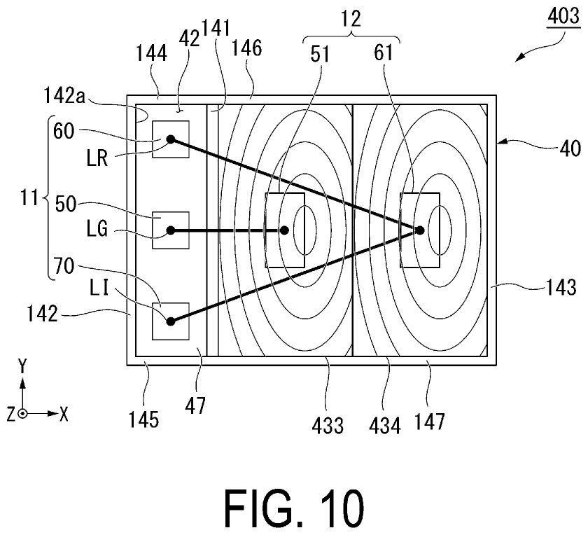

FIG. 10 is a plan view showing a main part configuration of a detection device 403 of the embodiment.

As shown in FIG. 10, the detection device 403 of the embodiment further includes the light emitting unit part 11, the light receiving unit part 12, the case member 40, the encapsulating layer 42, a first optical member 433, a second optical member 334, and the cover member 47.

The first optical member 433 and the second optical member 434 are configured of eccentric Fresnel lenses. Specifically, the first optical member 433 has a structure in which a center of grooves in which ellipses are concentrically arranged is shifted to the side (+X side) opposite to the light emitting unit part 11 in the X axis direction. The second optical member 334 has a structure in which a center of grooves in which ellipses are concentrically arranged is shifted to the side (+X side) opposite to the light emitting unit part 11 in the X axis direction.

That is, in the embodiment, center positions of the first light receiving part 51 and the first optical member 433 are different from each other, and center positions of the second light receiving part 61 and the second optical member 334 are different from each other.

In the detection device 403 of the embodiment, the green light LG, which is incident from the first light emitting part 50, propagates in the living body, and is radiated, is incident on the first optical member 433 from an oblique direction, but the green light LG is refracted toward the first light receiving part 51 side by the first optical member 433 configured of an eccentric Fresnel lens. Accordingly, the first light receiving part 51 can efficiently receive the green light LG radiated from the living body.

In addition, the red light LR, which is incident from the second light emitting part 60, propagates in the living body, and is radiated, is obliquely incident on the second optical member 334, but the red light LR is refracted toward the second light receiving part 61 side by the second optical member 334 configured of an eccentric Fresnel lens. Accordingly, the second light receiving part 61 can efficiently receive the red light LR radiated from the living body.

Similarly, the near-infrared light LI, which is incident from the third light emitting part 70, propagates in the living body, and is radiated, is incident on the second optical member 334 from an oblique direction, but the near-infrared light LI is refracted toward the second light receiving part 61 side by the second optical member 334 configured of an eccentric Fresnel lens. Accordingly, the second light receiving part 61 can efficiently receive the near-infrared light LI radiated from the living body.

As described above, according to the detection device 403 of the embodiment, by controlling the directivity of the light that enters the living body from the light emitting parts 50, 60, and 70, propagates in the living body, and is radiated, the light can be efficiently captured by the light receiving parts 51 and 61. Accordingly, according to the detection device 403 of the embodiment, it is possible to provide a detection device having high light use efficiency and high light reception accuracy.

Although the present disclosure has been described above based on the above-described embodiments, the present disclosure is not limited to the above embodiments and can be implemented in various aspects without departing from the gist thereof.

For example, in the above embodiments, the case in which the first wall part 141 of the case member 40 functions as a light blocking member has been described as an example, but the first wall part 141 may be configured to have light reflecting properties similarly to the second wall part 142. In this case, since the first wall part 141 reflects some of the light radiated from each light emitting part and causes it to enter the living body, it is possible to increase the light use efficiency of each light emitting part.

Also, in the above embodiments, a case in which the second light receiving part 61 receives both the red light LR and the near-infrared light LI has been described as an example, but a third light receiving part for receiving the near-infrared light LI may be provided separately. In this case, when an optical member is provided on the light receiving unit part 12 side, the near-infrared light LI is refracted by a third optical member disposed to correspond to the third light receiving part, whereby the near-infrared light LI can be efficiently incident on the third light receiving part.

Also, in the first embodiment, a case in which the first wall part 141, the second wall part 142, the fourth wall part 144, and the fifth wall part 145 are used to position the first optical member 33, the second optical member 34, and the third optical member 35 has been described as an example, but a method for positioning the first optical member 33, the second optical member 34, and the third optical member 35 is not limited thereto. For example, instead of using various wall members of the case member 40 for positioning, the first optical member 33 may be disposed to overlap the first light emitting region 140A1 with the first light emitting region 140A1 of the case member 40 as a reference, the second optical member 34 may be disposed to overlap the second light emitting region 140A2 with the second light emitting region 140A2 of the case member 40 as a reference, or the third optical member 35 may be disposed to overlap the third light emitting region 140A3 with the third light emitting region 140A3 of the case member 40 as a reference. In this case, each wall part does not need to protrude from the upper surface of the encapsulating layer 42.

Further, in the first embodiment, a case in which the first distance Dl between the first light emitting part 50 and the first light receiving part 51 is set to be smaller than the distance between the second light emitting part 60 or the third light emitting part 70 and the second light receiving part 61 (the second distance D2 or the third distance D3) has been described as an example, but the present disclosure is not limited thereto For example, the positions of the first light receiving part 51 and the second light receiving part 61 may be exchanged. That is, in the X axis direction, the distance from the first light emitting part 50 to the first light receiving part 51 may be longer than the distance from the second light emitting part 60 to the second light receiving part 61 or the distance from the third light emitting part 70 to the second light receiving part 61.

In this case, it is effective to make the focal length of the first optical member 33 longer than the focal length of the second optical member 34.

Thus, the first optical member 33 can cause the green light LG to enter deep into the living body while inhibiting attenuation of the green light LG by appropriately controlling the directivity of the green light LG. Accordingly, the first light receiving part 51 disposed farther than the second light receiving part 61 in the direction along the X axis can efficiently receive the green light LG that has propagated in the living body and radiated.

In addition, the second optical member 34 or the third optical member 35 can cause the red light LR and the near-infrared light LI to be incident on a position close to the surface layer in the living body by appropriately controlling the directivity of the red light LR and the near-infrared light LI. Accordingly, the second light receiving part 61 disposed near the second light emitting part 60 or the third light emitting part 70 in the direction along the X axis can efficiently receive the red light LR or the near-infrared light LI that has propagated in the living body and radiated.

Also, in the above embodiments, a person has been exemplified as the living body, but the present disclosure can also be applied to measurement of biological information (for example, pulse) of other animals.

In addition, in the measurement device 100 of the above embodiments, a case in which the detection device 3 is provided in the housing part 1 has been described as an example, but an installation location of the detection device 3 is not limited thereto and may be embedded in the belt 2, for example.

Further, a wristwatch type configuration has been exemplified as the measurement device 100 of the above embodiments, but the present disclosure can also be applied to, for example, a configuration to be worn around a subject's neck as a necklace type, a configuration to be attached to a subject's body as a sticker type, or a configuration to be attached to a subject's head as a head-mounted display type

Also, in the above embodiments, a case in which the bandpass filter 122 is provided only in the first light receiving part 51 has been described as an example, but a bandpass filter that selectively transmits the red light LR or the near-infrared light LI may also be provided in the second light receiving part 61.

Further, in the above embodiments, a case in which each of the light emitting parts 50, 60, and 70 is caused to emit light in a time division manner has been described as an example, but the first light receiving part 51 corresponding to the green light LG of the first light emitting part 50 is individually provided, and thus the first light emitting part 50 may be turned on all the time instead of in a time division manner.

A summary of the present disclosure will be appended below.

### Appendix 1

A detection device including:
a first light emitting part configured to emit first light having a first wavelength band toward a living body;
a second light emitting part configured to emit second light having a second wavelength band longer than the first wavelength band toward the living body;
a first light receiving part configured to receive the first light emitted from the first light emitting part and radiated from the living body;
a second light receiving part configured to receive the second light emitted from the second light emitting part and radiated from the living body;
a first optical member configured to refract the first light; and
a second optical member configured to refract the second light.

According to the detection device having this configuration, since the first optical member and the second optical member are individually provided to correspond to the first light and the second light, it is possible to individually optimize the directivity of the first light and the second light. Thus, the first light receiving part and the second light receiving part can detect the first light and the second light that have passed through the living body with high accuracy. Accordingly, it is possible to provide a highly reliable detection device having high light use efficiency of the first light emitting part and the second light emitting part and high detection accuracy.

### Appendix 2

The detection device according to Appendix 1, wherein
a focal length of the first optical member is shorter than a focal length of the second optical member, and
a distance from the first light emitting part to the first light receiving part in a second direction intersecting a first direction in which the first light emitting part and the second light emitting part are arranged is shorter than a distance from the second light emitting part to the second light receiving part.

According to this configuration, even when the first distance between the first light emitting part and the first light receiving part is shorter than the second distance between the second light emitting part and the second light receiving part, it is possible to individually optimize the directivity of the first light and the second light.

### Appendix 3

The detection device according to Appendix 1, wherein
a focal length of the first optical member is longer than a focal length of the second optical member, and
a distance from the first light emitting part to the first light receiving part in a second direction intersecting a first direction in which the first light emitting part and the second light emitting part are arranged is longer than a distance from the second light emitting part to the second light receiving part.

According to this configuration, even when the first distance between the first light emitting part and the first light receiving part is longer than the second distance between the second light emitting part and the second light receiving part, it is possible to individually optimize the directivity of the first light and the second light.

### Appendix 4

The detection device according to any one of Appendixes 1 to 3, further including a case member configured to hold the first light emitting part, the second light emitting part, the first light receiving part, and the second light receiving part, wherein,
when viewed, in plan view, in a third direction intersecting the first direction and the second direction,
the first optical member is disposed at a position at which the first optical member overlaps at least a part of a first light emitting region in which the first light emitting part of the case member is provided, and
the second optical member is disposed at a position at which the second optical member overlaps at least a part of a second light emitting region in which the second light emitting part of the case member is provided.

According to this configuration, the first optical member and the second optical member can efficiently receive the light emitted from the corresponding light emitting parts.

### Appendix 5

The detection device according to any one of Appendixes 1 to 4, wherein,
when viewed, in plan view, in the third direction,
at least either center positions of the first light emitting part and the first optical member or center positions of the second light emitting part and the second optical member are shifted from each other.

According to this configuration, by controlling the directivity of the light radiated from the light emitting parts whose center positions are shifted, the amount of light diffused in the direction opposite to the light receiving parts can be curbed. Accordingly, since the light radiated from the light emitting parts propagates in the living body and is efficiently incident on the light receiving parts, it is possible to provide a detection device having high light use efficiency and high light reception accuracy.

### Appendix 6

The detection device according to any one of Appendixes 1 to 5, wherein
the case member includes a first wall part provided between the first light emitting part and the first light receiving part in the second direction, and a second wall part provided on a side opposite to the first wall part with respect to the first light emitting part, and
the first optical member and the second optical member are held between the first wall part and the second wall part in the second direction.

According to this configuration, each of the first optical member and the second optical member is accurately positioned on the first light emitting part and the second light emitting part in the second direction.

### Appendix 7

The detection device according to any one of Appendixes 1 to 3, further including a case member configured to hold the first light emitting part, the second light emitting part, the first light receiving part, and the second light receiving part, wherein,
when viewed, in plan view, in a third direction intersecting the first direction and the second direction,
the first optical member is disposed at a position at which the first optical member overlaps at least a part of a first light receiving region in which the first light receiving part of the case member is provided, and
the second optical member is disposed at a position at which the second optical member overlaps at least a part of a second light receiving region in which the second light receiving part of the case member is provided.

According to this configuration, the first optical member and the second optical member can efficiently make the light incident on the light receiving parts by controlling the directivity of the corresponding light.

### Appendix 8

The detection device according to Appendix 7, wherein,
when viewed, in plan view, in the third direction,
at least either center positions of the first light receiving part and the first optical member or center positions of the second light receiving part and the second optical member are shifted from each other.

According to this configuration, by controlling the directivity of the light incident on the light receiving parts whose center positions are shifted, it is possible to efficiently make the light that has propagated in the living body incident on the light receiving parts.

### Appendix 9

The detection device according to Appendix 7 or 8, wherein
the case member includes a first wall part provided between the first light emitting part and the first light receiving part in the second direction, and a third wall part provided on a side opposite to the first wall part with respect to the first light receiving part, and
the first optical member and the second optical member are held between the first wall part and the third wall part in the second direction.

According to this configuration, each of the first optical member and the second optical member is accurately positioned on the first light receiving part and the second light receiving part in the second direction.

### Appendix 10

The detection device according to any one of Appendixes 7 to 9, wherein
when viewed, in plan view, in the third direction, an area of the first optical member is greater than an area of the first light receiving part, and an area of the second optical member is greater than an area of the second light receiving part.

According to this configuration, by condensing the first light or the second light using each optical member, it is possible to make the first light or the second light incident on the light receiving part having a small size.

### Appendix 11

The detection device according to any one of Appendixes 1 to 10, further including a third light emitting part configured to emit third light having a third wavelength band longer than the second light toward the living body, and a third optical member configured to refract the third light.

According to this configuration, since the third optical member individually provided to correspond to the third light is provided, it is possible to optimize the directivity of the third light.

### Appendix 12

The detection device according to any one of Appendixes 1 to 10, further including a third light emitting part configured to emit third light having a third wavelength band longer than the second light toward the living body, wherein
the second light receiving part receives the second light and the third light that are radiated from the living body and refracted by the second optical member.

According to this configuration, it is possible to detect the second light and the third light that have passed through the living body using the second light receiving part. Accordingly, since there is no need to separately provide a light receiving part for detecting the third light, it is possible to reduce the size of the detection device.

### Appendix 13

The detection device according to any one of Appendixes 1 to 12, further including a first wall part provided between the first light emitting part and the first light receiving part in the second direction, and a second wall part provided on a side opposite to the first wall part, wherein
at least either the first wall part or the second wall part reflects the first light emitted from the first light emitting part toward the first optical member.

According to this configuration, since the first light emitted from the first light emitting part is reflected and incident on the living body, it is possible to improve the light use efficiency of the first light emitting part.

### Appendix 14

The detection device according to Appendix 13, wherein
the first wall part absorbs the first light emitted from the first light emitting part.

According to this configuration, it is possible to inhibit a decrease in detection accuracy due to some of the first light radiated from the first light emitting part being directly incident on the first light receiving part or the second light receiving part.

### Appendix 15

The detection device according to any one of Appendixes 1 to 14, wherein
the first light receiving part includes an optical filter configured to transmit the first light and block the second light.

According to this configuration, by blocking the second light with the optical filter, it is possible to inhibit a decrease in detection accuracy due to the second light being incident on the first light receiving part.

### Appendix 16

A measurement device including:
the detection device according to any one of Appendixes 1 to 15; and
an information analysis unit configured to identify biological information from a detection signal indicating a detection result of the detection device.

According to the measurement device having this configuration, since a highly reliable detection device with high light use efficiency and high detection accuracy is provided, it is possible to provide a measurement device that enables highly accurate detection while inhibiting power consumption.

What is claimed is:

1. A detection device, comprising:
a first light emitting part that emits a first light having a first wavelength band toward a living body;
a second light emitting part that emits a second light having a second wavelength band longer than the first wavelength band toward the living body;
a first light receiving part that receives the first light emitted from the first light emitting part and radiated from the living body;
a second light receiving part that receives the second light emitted from the second light emitting part and radiated from the living body;
a first optical member that refracts the first light; and
a second optical member that refracts the second light.

2. The detection device according to claim 1, wherein
a focal length of the first optical member is shorter than a focal length of the second optical member, and
a distance from the first light emitting part to the first light receiving part is shorter than a distance from the second light emitting part to the second light receiving part, in a second direction intersecting a first direction in which the first light emitting part and the second light emitting part are arranged.

3. The detection device according to claim 1, wherein
a focal length of the first optical member is longer than a focal length of the second optical member, and
a distance from the first light emitting part to the first light receiving part is longer than a distance from the second light emitting part to the second light receiving part, in a second direction intersecting a first direction in which the first light emitting part and the second light emitting part are arranged.

4. The detection device according to claim 2, further comprising a case member that holds the first light emitting part, the second light emitting part, the first light receiving part, and the second light receiving part, wherein,
when viewed, in a plan view, in a third direction intersecting the first direction and the second direction,
the first optical member is disposed at a position at which the first optical member overlaps at least a part of a first light emitting region in which the first light emitting part of the case member is provided, and
the second optical member is disposed at a position at which the second optical member overlaps at least a part of a second light emitting region in which the second light emitting part of the case member is provided.

5. The detection device according to claim 4, wherein,
when viewed, in the plan view, in the third direction,
at least either center positions of the first light emitting part and the first optical member or center positions of the second light emitting part and the second optical member are different from each other.

6. The detection device according to claim 4, wherein
the case member includes a first wall part provided between the first light emitting part and the first light receiving part in the second direction, and a second wall part provided on a side opposite to the first wall part with respect to the first light emitting part, and
the first optical member and the second optical member are held between the first wall part and the second wall part in the second direction.

7. The detection device according to claim 2, further comprising a case member that holds the first light emitting part, the second light emitting part, the first light receiving part, and the second light receiving part, wherein,
when viewed, in a plan view, in a third direction intersecting the first direction and the second direction,
the first optical member is disposed at a position at which the first optical member overlaps at least a part of a first light receiving region in which the first light receiving part of the case member is provided, and
the second optical member is disposed at a position at which the second optical member overlaps at least a part of a second light receiving region in which the second light receiving part of the case member is provided.

8. The detection device according to claim 7, wherein,
when viewed, in the plan view, in the third direction,
at least either center positions of the first light receiving part and the first optical member or center positions of the second light receiving part and the second optical member are different from each other.

9. The detection device according to claim 7, wherein
the case member includes a first wall part provided between the first light emitting part and the first light receiving part in the second direction, and a third wall part provided on a side opposite to the first wall part with respect to the first light receiving part, and
the first optical member and the second optical member are held between the first wall part and the third wall part in the second direction.

10. The detection device according to claim 7, wherein
when viewed, in the plan view, in the third direction, an area of the first optical member is greater than an area of the first light receiving part, and an area of the second optical member is greater than an area of the second light receiving part.

11. The detection device according to claim 2, further comprising a third light emitting part that emits a third light having a third wavelength band longer than the second light toward the living body, and a third optical member configured to refract the third light.

12. The detection device according to claim 7, further comprising a third light emitting part that emits a third light having a third wavelength band longer than the second light toward the living body, wherein
the second light receiving part receives the second light and the third light that are radiated from the living body and refracted by the second optical member.

13. The detection device according to claim 2, further comprising a first wall part provided between the first light emitting part and the first light receiving part in the second direction, and a second wall part provided on a side opposite to the first wall part, wherein
at least either the first wall part or the second wall part reflects the first light emitted from the first light emitting part toward the first optical member.

14. The detection device according to claim 13, wherein
the first wall part absorbs the first light emitted from the first light emitting part.

15. The detection device according to claim 2, wherein
the first light receiving part includes an optical filter configured to transmit the first light and block the second light.

16. A measurement device, comprising:
the detection device according to claim 1; and
an information analysis part configured to identify biological information from a detection signal indicating a detection result of the detection device.
